(19) United States
(12) Patent Application Publication
(10) Pub. No.: US 2020/0329884 A1
(43) Pub. Date: Oct. 22, 2020

(54) Biofeedback Chair System

(71) Applicant: Kaleb Matson, Denver, CO (US)

(72) Inventor: Kaleb Matson, Denver, CO (US)

(73) Assignee: Lumena Inc, Denver, CO (US)

(21) Appl. No.: 16/851,022

(22) Filed: Apr. 16, 2020

Related U.S. Application Data
(60) Provisional application No. 62/834,402, filed on Apr. 16, 2019.

(51) Int. Cl. A47C 31/12; A47C 7/72; G06F 3/01; G02B 27/01; A61B 5/01; A61B 5/0476; A61B 5/0402

(52) U.S. Cl. CPC A47C 31/126; A47C 7/727; G06F 3/015; A61B 5/0402; G02B 27/017; A61B 5/01; A61B 5/0476; G06F 3/016

(57) ABSTRACT

An interactive chair with an optional headset comprising integrated electrocardiogram and electroencephalography sensors and pulsed electromagnetic field generators along with other input and outputs necessary for a user's experience to be manipulated by a computer from the user's present state, ab initio, or from the user's present state in conjunction with other media; thereby allowing a user of the chair to integrate a wide variety of physical and electromagnetic biofeedback experiences into their daily lives. When used in conjunction with media, the chair allows the user to experience media in a manner that is not predetermined by the media alone, but also by the user's current physical experience; thus, adding additional variable dimensions to the experience of the media.

### CROSS REFERENCE TO RELATED APPLICATIONS

This application claims the benefit of U.S. Provisional Patent Application No. 62/834,402, filed on Apr. 16, 2020, and titled “Pulsed Electro Magnetic Field Chair” which is incorporated by reference herein in its entirety for all purposes.

### BACKGROUND OF THE INVENTION

### 1. Field of the Invention

The present invention is in the technical field of interactive seating; more particularly, the present system pertains to an interactive pulsed electromagnetic field chair system.

### 2. Description of Related Art

As with almost all inventions, much of what is disclosed herein is a combination of elements that have been done before individually; what is unique and valuable is the specific combination of elements and what that combination of elements allows the user to understand, experience, and control. Interactive chairs are well known in the art, chairs that have speakers, rockers, massager, and heaters are all familiar in the art, as are virtual reality headsets with screens and headphones. Likewise, in non-chair related arts infrared generators, lasers, LEDS, and pulsed electromagnetic field generators are well-known in the art. In scientific and medical communities, sensors such as heart rate monitors, blood pressure monitors, breath rate monitors, electrocardiogram monitors, electroencephalography monitors are well known in the art.

The problem until now has been that these various systems have not been combined in a way that allows for them to be used in combination while also being built into a chair, so that the chair communicates the activity of the chair with a computer and the computer can then return communication to the user through the chair.

There have been massage chairs that integrate the user's pulse rate into the massage experience, with the present disclosure, users of chairs will now be able integrate a wide variety of physical and electromagnetic biofeedback experiences into their daily lives.

The internet of chairs and custom electromagnetic biofeedback experiences allows users to have a tailored experience either ab initio, or in conjunction with media, in a manner that is not predetermined by the media alone, but also by the user's current physical experience; thus adding additional variable dimensions to the experience. For example, the chair could relax you in the evenings before bed and it could stimulate you during the day; or it could be set to stimulate uneasiness and tension during climactic portions of a movie based on correlation between the user's current conditions and certain preset conditions in the media; and then.

### SUMMARY

The scope of the present invention is defined solely by the appended claims and detailed description of a preferred embodiment and is not affected to any degree by the statements within this summary. Generally, a chair with an optional headset is described wherein the chair and headset comprise various sensors and generators of stimulations, most importantly electrocardiogram and electroencephalography sensors and pulsed electromagnetic field generators.

The interactive pulsed electromagnetic field chair system provides the input and output equipment necessary for a user's experience to be manipulated by a computer from the user's present state, ab initio, or from a user's present state in conjunction with other media. When used in conjunction with media, the chair allows the user to experience media in a manner that is not predetermined by the media alone, but also by the user's current physical experience; thus, adding additional variable dimensions to the experience of the media. Essentially, a user of the chair will be able integrate a wide variety of physical and electromagnetic biofeedback experiences into their daily lives.

### BRIEF DESCRIPTION OF THE DRAWINGS

Various embodiments are described herein with reference to the following Drawings. Certain aspects of the Drawings are depicted in a simplified way for reason of clarity. Not all alternatives and options are shown in the Drawings and, therefore, the Claims are not limited in scope to the content of the Drawings.

### 1. FIGURES

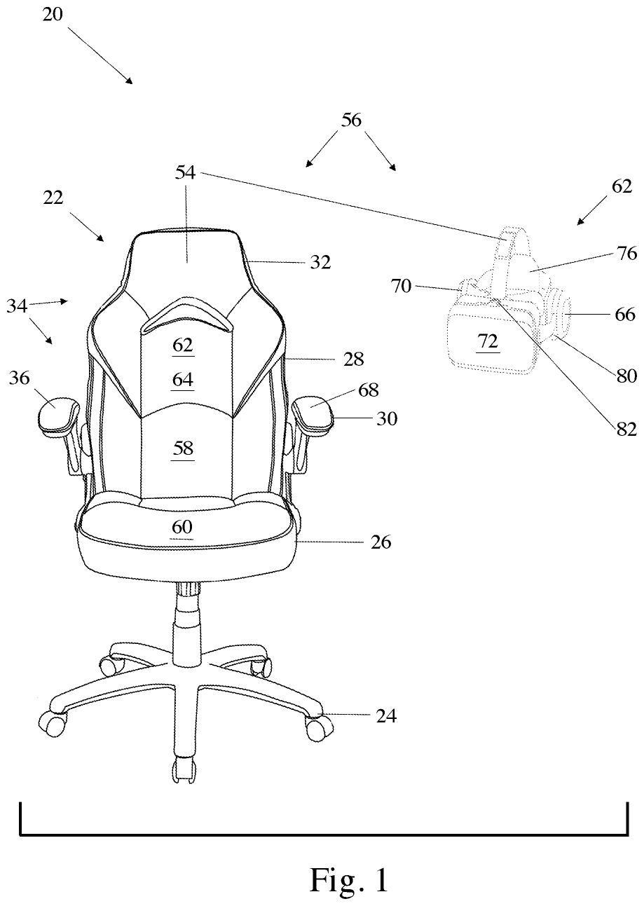

FIG. 1 illustrates a front view of an interactive pulsed electromagnetic field chair system, in accordance with an embodiment of the present disclosure.

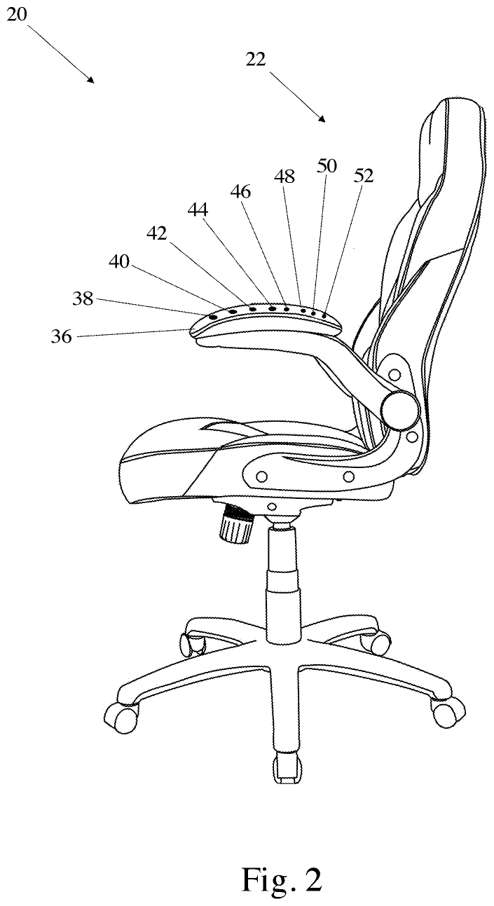

FIG. 2 illustrates a side view of the chair of an interactive pulsed electromagnetic field chair system, in accordance with an embodiment of the present disclosure.

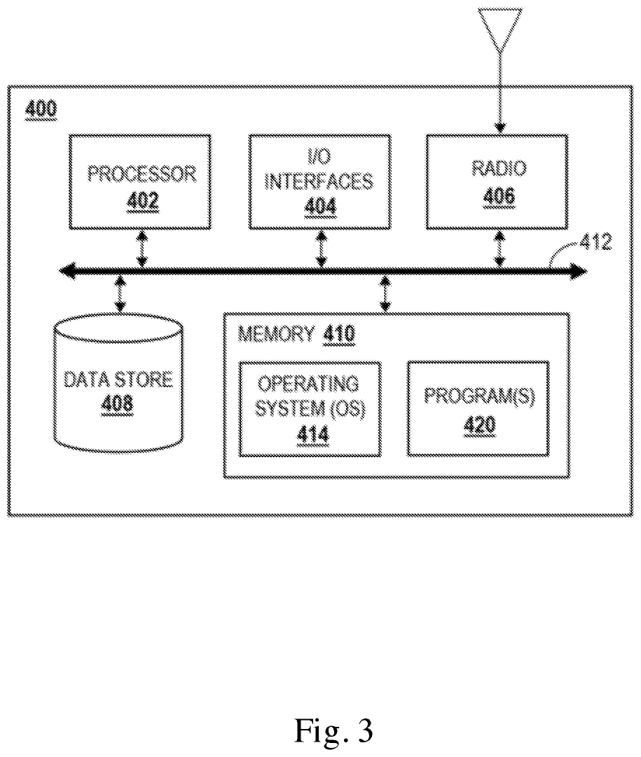

FIG. 3 illustrates method of using an interactive pulsed electromagnetic field chair system in accordance with an embodiment of the present disclosure.

Corresponding reference characters indicate corresponding components throughout the several figures of the Drawings. Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood elements that are useful or necessary in commercially feasible embodiment are often not depicted in order to facilitate a less obstructed view of these various embodiments of the present disclosure.

### 2. REFERENCES

20 Interactive Pulsed Electromagnetic Field Chair System
22 Chair
24 Chair Base
26 Chair Seat
28 Chair Back
30 Chair Arms
32 Chair Headrest
34 Output Sensors
36 Biosensor Strip
38 Heart Rate Sensor
40 Heart Rate Variability Sensor
42 Pulse Oximeter Sensor
44 Breath Rate Sensor
46 Blood Pressure Sensor
48 Electrocardiogram (EKG) Sensor
50 Body Temperature Sensor
52 Electrodermal Activity (EDA) Sensor
54 Electroencephalography (EEG) Sensor
56 Physical Input Generators
58 Pulsed Electromagnetic Field (PEMF) Generators
60 Tactile Transducers
62 Mid, Near, And Far Infrared Generators
64 Ultrasound Generator
66 Speakers
68 Light Source
70 Headset
72 Virtual Reality Screen
74 Headphones
76 Transcranial Direct-Current Generator
78 Headset LEDs
80 Headset PEMF Coils
82 Aromatherapy Diffusers
400 Client Device
402 Processor
404 I/O Interfaces
406 Radio
408 Data Store
410 Memory
412 Local Interface
414 Operating System (OS)
420 Program(s)

### DETAILED DESCRIPTION

The following description is not to be taken in a limiting sense but is made merely for the purpose of describing the general principles of exemplary embodiments, many additional embodiments of this invention are possible. It is understood that no limitation of the scope of the invention is thereby intended. The scope of the disclosure should be determined with reference to the Claims. Reference throughout this specification to “one embodiment,” “an embodiment,” or similar language means that a particular feature, structure, or characteristic that is described in connection with the embodiment is included in at least one embodiment of the present disclosure. Thus, appearances of the phrases “in one embodiment,” “in an embodiment,” and similar language throughout this specification may, but do not necessarily, all refer to the same embodiment.

Further, the described features, structures, or characteristics of the present disclosure may be combined in any suitable manner in one or more embodiments. In the Detailed Description, numerous specific details are provided for a thorough understanding of embodiments of the disclosure. One skilled in the relevant art will recognize, however, that the embodiments of the present disclosure can be practiced without one or more of the specific details, or with other methods, components, materials, and so forth. In other instances, well-known structures, materials, or operations are not shown or described in detail to avoid obscuring aspects of the present disclosure. Any alterations and further modifications in the illustrated devices, and such further application of the principles of the invention as illustrated herein are contemplated as would normally occur to one skilled in the art to which the invention relates.

The phrases “at least one,” “one or more,” and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. The terms “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

As used herein, the term “computer” refers to a machine, apparatus, or device that is capable of accepting and performing logic operations from software code. The term “application”, “software”, “software code”, “source code”, “script”, or “computer software” refers to any set of instructions operable to cause a computer to perform an operation. Software code may be operated on by a “rules engine” or processor. Thus, the methods and systems of the present invention may be performed by a computer or computing device having a processor based on instructions received by computer applications and software.

The term “electronic device” as used herein is a type of computer comprising circuitry and configured to generally perform functions such as recording, displaying, or reproducing electronic data; providing electrical communications and network connectivity; or any other similar function. Non-limiting examples of electronic devices include: personal computers (PCs), servers, laptops, tablet PCs including the iPad, smart phones, or any electronic device capable of running computer software and displaying information to a user. Certain types of electronic devices which are portable and easily carried by a person from one location to another may sometimes be referred to as a “portable electronic device” or “portable device”.

The term “client device” as used herein is a type of computer or computing device comprising circuitry and configured to generally perform functions such as recording signal data from output sensors, and storing, retrieving, or processing of electronic data; providing electrical communications and network connectivity; and sending control signals to physical input generators or any other similar function. Non-limiting examples of client devices include: personal computers (PCs), servers, laptops, tablet PCs, smart phones, or any electronic device capable of running computer software and displaying information to a user.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The present disclosure is to be considered as an exemplification of the invention and is not intended to limit the invention to the specific embodiments illustrated by the figures or description below. Generally, the described invention relates to a novel combination of output sensors and physical input generators integrated into a chair and optional headset; secondarily, a system for using said inputs and outputs is disclosed.

FIG. 1 illustrates a front view of an interactive pulsed electromagnetic field chair system 20, in accordance with an embodiment of the present disclosure. Generally, the system 20 comprises a chair 22; but in some embodiments it may also comprise a headset 62. The chair and headset may both comprise various monitoring sensors that create one or more output signals related to a user that are in communication with a computer; additionally, the two primary structures both comprise various generators of user stimulation that receive one or more input signals related to a user that are also in communication with a computer. The chair 22 and possibly the headset 62, in combination, then provide the input and output apparatus necessary for a user's experience to be manipulated by a computer from a user's present state, ab initio, or from the user's present state in conjunction with other media. When used in conjunction with media, the chair allows a user to experience media in a manner that is not predetermined by the media alone, but also by the user's current physical experience; thus, adding additional variable dimensions to the experience of the media.

As seen in FIG. 1 the chair 22 may comprise the standard elements of a chair: a chair base 24, a chair seat 26, a chair back 28, chair arms 30, and a chair headrest 32. In the preferred embodiment, most of the features of the chair are built into the chair and would not be visible from the exterior of the chair. The chair 22 comprises one or more output sensors 34. As seen in FIG. 1 the output sensors 34 are preferably located on the arm of the chair where they may come into direct contact with the skin of the arm and hands of a user and get their most accurate readings and arranged in either one or two biosensor strips 36. The chair may comprise one or more of the following sensors: heart rate sensor 38, heart rate variability sensor 40, pulse oximeter sensor 42, breath rate sensor 44, blood pressure sensor 46, electrocardiogram (EKG) sensor 48, body temperature sensor 50, and/or electrodermal activity (EDA) sensor 52 may be located in either the chair 22 or an optional headset 70. FIG. 2 illustrates an embodiment of a side view of an interactive pulsed electromagnetic field chair system 20 wherein the various output sensors 34 and biosensor strip 36 along the arm of the chair 30 are visible. Additionally, an optional electroencephalography (EEG) Sensor 54 may be located in the headrest of the chair 32 but is preferably in the optional headset 70. The various output sensors 34 of the chair 22 monitor a user and create a signal which is in communication with a computer which may record and process the signals; then sending a return signal to the physical input generators 56, described below.

The interactive pulsed electromagnetic field chair system 20 also comprises one or more of the following physical input generators 56 that modify the experience of the user of the chair according to their function: pulsed electromagnetic field (PEMF) generator 58, tactile transducer 60, mid, near, and far infrared generator 62, ultrasound generator 64, speaker 66, light sources 68, virtual reality screen 72, headphones 74, transcranial direct-current generator 76, and/or aromatherapy diffusers 82.

The following is a description of the layout of the physical input generators in the preferred embodiment: one pulsed electromagnetic field (PEMF) generator 58 is built into the seat of the chair 26, two are built into the back of the chair 28, and two are built into the optional headset 80 or the headrest 32 of the chair; one tactile transducer is built into the seat of the chair 26, two are built into the back of the chair 28; a combination panel of mid, near, and far infrared generators 62 are built into the back of the chair 28, along with an ultrasound generator 64; two speakers 66 are built into the optional headphones 74 or the headrest 32 of the chair; light sources 68 are built into the arms 30 of the chair with six on each side and also into the headset 70 along with the virtual reality screen 72, transcranial direct-current generator 76, and/or aromatherapy diffusers 82. The cushions of the chair itself may be made of a gel to improve the transmission of vibrations from the transducers.

In some embodiments, a light source 68 may comprise a light emitting diode (LED) which may be configured to provide light of various wavelengths and intensities, such as: 626 nm—red, 470 nm—blue, 560 nm—green, 850 nm—infrared. In other embodiments, a light source 68 may comprise a laser light emitter which may be optionally motorized for directing the laser light in various directions and motion patterns. In still further embodiments, a light source 68 may comprise an organic light-emitting diode (OLED), incandescent light bulb, fluorescent light, bulb halogen light bulb, high-intensity discharge light bulb, electroluminescent light source, neon light source, or light strips.

Said pulsed electromagnetic field (PEMF) generator may generate either static and/or pulsed, delivering frequencies on the spectrum from 0 hz to 10,000 hz, to the body. The pulsed electro-magnetic fields device 58 may also be designed to create three or more frequencies at once to create a complex harmonic magnetic field.

The chair itself may be in an enclosed space, the enclosed space may have non-native EMF shielding (which may essentially a Faraday Cage which filters/attenuates the spectrum of man and electro-magnetic fields). The enclosed space may also be mirrored on the walls, ceiling, and floor.

Referring now to FIG. 3, in an exemplary embodiment, a block diagram illustrates a client device 400 of which one or more may be used with the interactive pulsed electromagnetic field chair system 20 and which may be a type of computing platform. The client device 400 can be a digital device that, in terms of hardware architecture, generally includes a processor 402, input/output (I/O) interfaces 404, a radio 406, a data store 408, and memory 410. It should be appreciated by those of ordinary skill in the art that FIG. 3 depicts the client device 400 in an oversimplified manner, and a practical embodiment may include additional components and suitably configured processing logic to support known or conventional operating features that are not described in detail herein. The components (402, 404, 406, 408, and 410) are communicatively coupled via a local interface 412.The local interface 412 can be, for example but not limited to, one or more buses or other wired or wireless connections, as is known in the art. The local interface 412 can have additional elements, which are omitted for simplicity, such as controllers, buffers (caches), drivers, repeaters, and receivers, among many others, to enable communications. Further, the local interface 412 may include address, control, and/or data connections to enable appropriate communications among the aforementioned components.

The processor 402 is a hardware device for executing software instructions. The processor 402 can be any custom made or commercially available processor, a central processing unit (CPU), an auxiliary processor among several processors associated with the client device 400, a semiconductor-based microprocessor (in the form of a microchip or chip set), or generally any device for executing software instructions. When the client device 400 is in operation, the processor 402 is configured to execute software stored within the memory 410, to communicate data to and from the memory 410, and to generally control operations of the client device 400 pursuant to the software instructions. In an exemplary embodiment, the processor 402 may include a mobile optimized processor such as optimized for power consumption and mobile applications.

The I/O interfaces 404 can be used to receive data signals from the output sensors 34 of the chair 22 and/or for sending control signals to the physical input generators 56, including the pulsed electromagnetic field (PEMF) generators 58. Output sensor 34 signals can be provided via a plurality of I/O interfaces 404, and may be provided by one or more of the following sensors: heart rate sensor 38, heart rate variability sensor 40, pulse oximeter sensor 42, breath rate sensor 44, blood pressure sensor 46, electrocardiogram (EKG) sensor 48, body temperature sensor 50, electrodermal activity (EDA) sensor 52, and/or an electroencephalography (EEG) Sensor 54. Said output sensors 34 may send signals to a client devise 400 and said client devise 400 processes 402 said signals and sends a control signal to said physical input generators 56 that is related to said output sensor signals 34

System output can be provided via a display screen such as a liquid crystal display (LCD), touch screen, and the like. The I/O interfaces 404 can also include, for example, a global positioning service (GPS) radio, a serial port, a parallel port, a small computer system interface (SCSI), an infrared (IR) interface, a radio frequency (RF) interface, a universal serial bus (USB) interface, and the like. The I/O interfaces 404 can include a graphical user interface (GUI) that enables a user to interact with the client device 400. Additionally, the I/O interfaces 404 may be used to output control signals to the physical input generators 56, and may be provided by one or more of the following physical input generators: pulsed electromagnetic field (PEMF) generator 58, tactile transducer 60, mid, near, and far infrared generator 62, ultrasound generator 64, speaker 66, light sources 68, virtual reality screen 72, headphones 74, transcranial direct-current generator 76, and/or aromatherapy diffusers 82.

The radio 406 enables wireless communication to an external access device or network. Any number of suitable wireless data communication protocols, techniques, or methodologies can be supported by the radio 406, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Frequency Hopping Spread Spectrum; Long Term Evolution (LTE); cellular/wireless/cordless telecommunication protocols (e.g. 3G/4G, etc.); wireless home network communication protocols; paging network protocols; magnetic induction; satellite data communication protocols; wireless hospital or health care facility network protocols such as those operating in the WMTS bands; GPRS; proprietary wireless data communication protocols such as variants of Wireless USB; and any other protocols for wireless communication.

The data store 408 may be used to store data and is therefore a type of memory. The data store 408 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 408 may incorporate electronic, magnetic, optical, and/or other types of storage media.

The memory 410 may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 410 may incorporate electronic, magnetic, optical, and/or other types of storage media. Note that the memory 410 may have a distributed architecture, where various components are situated remotely from one another, but can be accessed by the processor 402. The software in memory 410 can include one or more software programs 420, each of which includes an ordered listing of executable instructions for implementing logical functions. In the example of FIG. 15, the software in the memory system 410 includes a suitable operating system (O/S) 414 and programs 420.

The operating system 414 essentially controls the execution of other computer programs, and provides scheduling, input-output control, file and data management, memory management, and communication control and related services. The operating system 414 may be, for example, LINUX (or another UNIX variant), Android, Symbian OS, Microsoft Windows CE, Microsoft Windows 7 Mobile, Microsoft Windows, iOS, and the like.

The programs 420 may include various applications, add-ons, etc. configured to provide end user functionality with the client device 400. For example, exemplary programs 420 may include, but not limited to, a web browser, social networking applications, streaming media applications, games, mapping and location applications, electronic mail applications, financial applications, and the like.

Information as herein shown and described in detail is fully capable of attaining the above-described object of the present disclosure, the presently preferred embodiment of the present disclosure; and is, thus, representative of the subject matter; which is broadly contemplated by the present disclosure. The scope of the present disclosure fully encompasses other embodiments which may become obvious to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims.

Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the present disclosure.

What is claimed is:

1. A chair, comprising:
a chair base;
a chair seat;
a chair back;
chair arms;
output sensors;
physical input generators;
wherein said output sensors send signals to a client devise and said client devise processes said signals and sends a control signal to said physical input generators that is related to said output sensor signals.

2. The chair of claim 1, further comprising a headset.

3. The chair of claim 2, wherein said headset is a virtual reality headset.

4. The chair of claim 2, wherein said electroencephalography (EEG) sensor is in said headset and not in said chair.

5. The chair of claim 1, wherein said signals are radio.

6. The chair of claim 1, wherein at least one of said output sensors is an electroencephalography (EEG) sensor

7. The chair of claim 1, wherein said output sensors further comprises a heart rate sensor.

8. The chair of claim 1, wherein said output sensors further comprises a heart rate variability sensor.

9. The chair of claim 1, wherein said output sensors further comprises a pulse oximeter sensor.

10. The chair of claim 1, wherein said output sensors further comprises a breath rate sensor.

11. The chair of claim 1, wherein said output sensors further comprises a blood pressure sensor.

12. The chair of claim 1, wherein said output sensors further comprises an electrocardiogram (EKG) sensor.

13. The chair of claim 1, wherein said output sensors further comprises a body temperature sensor.

14. The chair of claim 1, wherein said output sensors further comprises an electrodermal activity (EDA) sensor.

15. The chair of claim 1, wherein at least one of said physical input generators is a pulsed electromagnetic field (PEMF) generator

16. The chair of claim 1, wherein said input physical generators further comprise a tactile transducer.

17. The chair of claim 1, wherein said input physical generators further comprise an infrared generator.

18. The chair of claim 1, wherein said input physical generators further comprise an ultrasound generator.

19. The chair of claim 1, wherein said input physical generators further comprise speakers.

20. The chair of claim 1, wherein said input physical generators further comprise light sources.

21. The chair of claim 1, wherein said input physical generators further comprise transcranial direct-current generators.

22. The chair of claim 1, wherein said input physical generators further comprise a transcranial direct-current generator.

23. The chair of claim 1, wherein said input physical generators further comprise aromatherapy diffusers.
